(19) United States
(12) Patent Application Publication
(10) Pub. No.: US 2022/0000651 A1
(43) Pub. Date: Jan. 6, 2022

(54) ORTHOSIS WITH TEXTURED SURFACE

(71) Applicant: Kelly Robb, Woodstock (CA)

(72) Inventor: Kelly Robb, Woodstock (CA)

(21) Appl. No.: 16/920,575

(22) Filed: Jul. 3, 2020

(51) Int. Cl. A61F 5/14; A43B 17/00; A43D 29/00

(52) U.S. Cl. CPC A61F 5/14; A43D 29/00; A43B 17/006

(57) ABSTRACT

There is provided an orthosis and a method of making same. The orthosis includes a contoured insole having a medial side portion, the medial side portion having a contoured arch support portion. The orthosis further includes a top layer including a top surface having texture formations formed within one or more regions associated with the top surface, the texture formations having an orientation, wherein the orientation is dependent on an intended movement of a user during use of the orthosis.

### FIELD OF THE INVENTION

The present invention pertains to the field of orthoses and in particular to an orthosis with a textured surface.

### BACKGROUND

Muscles may not always function as desired. The optimization of muscle activity is desired across many movement related behaviours. These movement related behaviours can include locomotion (walking and/or running), sporting activities and activities which occur during day-to-day functioning. A decreased in sensory feedback can have a direct effect, for example through neurological pathways, on the proper functioning of the muscles of legs and feet, applicable to healthy young, healthy old and diseased populations. Impaired sensory function, irrespective of its cause, can effect normal movement. It is known that sensory feedback can be important in weight bearing movement. Interventions that are designed to improve sensory information can have the ability to modify the operation of muscles during these activities. This intervention may address multiple objectives, for example an increase in sensory information can signal a quicker onset of muscle activity and can improve muscle efficiency during weight bearing tasks.

Furthermore, athletes are continually striving to perform better, faster, and more efficiently in their desired sport. Therefore adding a stimulus mechanism under the foot, with a desire of triggering a quicker onset of muscle activity, has the potential to improve athletic performance. Secondly, a muscle that can perform the same work under less demand, may increase muscle performance while reducing long-term muscle fatigue.

In addition, sensory impairment is a complication to many neurological conditions, including Parkinson's disease & Multiple Sclerosis. Parkinson's disease is a common neurodegenerative disorder, with over 100,000 Canadians living with Parkinson's disease. In the United States, it is estimated that 930,000 people are living with Parkinson's disease. Similarly, there is an estimated 77,000 Canadians, and over 1 million Americans living with Multiple Sclerosis.

There are four types of receptors (cutaneous mechanoreceptors) located in the skin of the foot. These receptors have a function to sense change, from center of pressure and/or environmental change, and convey a response to the neurological system. The neurological system conveys this information to the spinal cord, which subsequently communicates with nerves delivering a motor response. The most populated receptor in the bottom of the feet are fast adapting type 1 (FA1). FA1 receptors preferentially respond to lateral skin deformation and stretch. Impairments to mechanoreceptor function decreases the ability to respond to environmental changes under the foot. These impairments can include a delay in receptor response, or an increase in the perceptual threshold required to sense a need to respond.

Currently, some insole designs intending to improve sensory feedback rely on electrical stimulation and/or vibration under the foot sole. Although these designs may prove effective, adding battery packs and the need to keep electrical charge in the batteries cause challenges to long-term wear.

U.S. Patent Application No. 2010/0269244 discloses an insole which includes a lateral side and a medial side wherein one of the lateral or medial sides of the insole has a smooth surface while the opposite side has a textured surface.

Accordingly, there may be a need for an apparatus for improving plantar sensory input that is not subject to one or more limitations of the prior art.

This background information is intended to provide information that may be of possible relevance to the present invention. No admission is necessarily intended, nor should be construed, that any of the preceding information constitutes prior art against the present invention.

### SUMMARY

It is an object of the present invention to obviate or mitigate at least one disadvantage of the prior art.

According to another aspect of the present invention there is provided an orthosis. The orthosis includes a contoured insole having a medial side portion, the medial side portion having a contoured arch support portion. The orthosis further includes a top layer including a top surface having texture formations formed within one or more regions associated with the top surface, the texture formations having an orientation, wherein the orientation is dependent on an intended movement of a user during use of the orthosis. The bottom surface of the top layer is coupled to a top surface of the contoured insole.

In some embodiments, the one or more patterns include a zigzag pattern, star shaped pattern, plus sign pattern, sinusoidal shaped pattern, a wave shaped pattern, chevron shaped pattern and a herringbone shaped pattern.

In some embodiments, the one or more regions include one or more of a forefoot region, a midfoot region and a rearfoot or heel region. In some embodiments, each of the forefoot region, the midfoot region and the rearfoot or heel region are defined by two regions including a medial regarding and a lateral region.

In some embodiments, the orientation of the texture formations is perpendicular to the intended movement of the user during use of the orthosis. In some embodiments, the orientation of the texture formations is parallel to the intended movement of the user during use of the orthosis.

According to an aspect of the present invention, there is provided a method for making an orthosis. The method includes forming a contoured insole having a medial side portion, the medial side portion having a contoured arch support portion. The method further includes forming a top layer, the top layer including a top surface having texture formations formed within one or more regions associated with the top surface, the texture formations having an orientation, wherein the orientation is dependent on an intended movement of a user during use of the orthosis. In addition, the method includes attaching a bottom surface of the top layer to a top surface of the contoured insole.

In some embodiments, forming one or more of the contoured insole and the top layer is performed using three dimensional printing. In some embodiments, forming the top layer including forming the texture formations using heat moulding or injection moulding or stamping. In some embodiments, the texture formations are formed using direct milling.

Embodiments have been described above in conjunction with aspects of the present invention upon which they can be implemented. Those skilled in the art will appreciate that embodiments may be implemented in conjunction with the aspect with which they are described but may also be implemented with other embodiments of that aspect. When embodiments are mutually exclusive, or are otherwise incompatible with each other, it will be apparent to those skilled in the art. Some embodiments may be described in relation to one aspect, but may also be applicable to other aspects, as will be apparent to those of skill in the art.

### BRIEF DESCRIPTION OF THE FIGURES

Further features and advantages of the present invention will become apparent from the following detailed description, taken in combination with the appended drawings, in which:

FIG. 1 illustrates a perspective view of a textured orthosis for a left foot according to embodiments.

FIG. 2A illustrates a perspective view of a forefoot regionally textured orthosis for a right foot according to embodiments.

FIG. 2B illustrates a perspective view of a forefoot regionally textured orthosis for a right foot according to embodiments.

FIG. 2C illustrates a perspective view of a forefoot regionally textured orthosis for a right foot according to embodiments.

FIG. 2D illustrates a perspective view of a forefoot regionally textured orthosis for a right foot according to embodiments.

FIG. 3A illustrates a perspective view of a midfoot regionally textured orthosis for a right foot according to embodiments.

FIG. 3B illustrates a perspective view of a midfoot regionally textured orthosis for a right foot according to embodiments.

FIG. 3C illustrates a perspective view of a midfoot regionally textured orthosis for a right foot according to embodiments.

FIG. 4A illustrates a perspective view of a rearfoot regionally textured orthosis for a right foot according to embodiments.

FIG. 4B illustrates a perspective view of a rearfoot regionally textured orthosis for a left foot according to embodiments.

FIG. 4C illustrates a perspective view of a rearfoot regionally textured orthosis for a left foot according to embodiments.

FIG. 5 illustrates a rear view of a rearfoot regionally textured orthosis for a right foot according to embodiments.

FIG. 6A illustrates a side view of a textured orthosis for a left foot according to embodiments.

FIG. 6B illustrates a perspective view of a textured orthosis for a left foot according to embodiments.

FIG. 7 illustrates a method for making a textured orthosis according to embodiments.

### DETAILED DESCRIPTION

It has been realised that impaired sensory function, irrespective of its cause, largely effects normal locomotion. Using sensory feedback can be important in the placement of each step, in the adaptation to uneven terrain, and in effectively using musculature during walking. Decreased sensory feedback has a direct effect (through neurological pathways) to the proper functioning of the muscles of legs and feet. As such, there is a need for an orthosis, that is easily worn during activities of daily living, which enhances sensory feedback to the neurological system, and can be further used across plural foot types. This format of orthosis may be effective in improving muscular response while reducing the need for wearing bulky bracing apparatuses.

There is provided an apparatus and method of making an orthosis to enhance the sensory feedback from the cutaneous mechanoreceptors located in the skin of the plantar foot sole. Adding texture under the foot can serve to create a continual stimulus to these receptors and enhance the speed and magnitude of available response to the nervous system. The orthosis of the instant disclosure is configured to enhance the sensory feedback and which may improve the conductance of information from sensory receptors in the feet to the nerves of one or both of the upper and lower leg.

According to embodiments, there are provided orthoses that include substantially full foot contact, which may be configured as custom made foot orthosis designs or standard designs for selected foot configurations, for example different levels of arch support. Embodiments are directed to textured foot orthoses, the configuration of the textured material, and the orientation of the texture associated with the orthosis.

There is provided an orthosis that includes a contoured insole having a medial side portion, the medial side portion having a contoured arch support portion. The orthosis further includes a top layer including a top surface having texture formations formed within one or more regions associated with the top surface. The texture formations have an orientation, wherein the orientation is dependent on an intended movement of a user during use of the orthosis. The bottom surface of the top layer is coupled to a top surface of the contoured insole thereby forming the orthosis.

According to some embodiments, the orthosis is configured to make substantially full foot contact with the plantar foot sole. In some embodiments, the orthosis is manufactured such that it is custom sized and configured to a foot shape of an intended user. In addition, the medial aspect of the orthosis, for example the arch support can be shaped to the wearer's foot. The textured material shall act as a top cover in the orthosis design, whereby the material contours the shape of the orthosis and wearer's plantar foot sole.

According to embodiments, the contoured arch support portion can be custom made for an individual, for example through the creation of a 3D foot impression. In other embodiments, the contoured arch support portion can be selected for an individual based on a generic arch shape. The generic arch shape may be divided into multiple versions thereof, which can account for a level of variability in arches of different individuals. For example, the generic arch shape can have arch height versions available to accommodate high, medium, or low arched foot postures. The arch support can provide a level of stabilization of the foot and ankle and can balance the effect of the further features of the orthosis which may be configured to improve plantar sensory input.

According to some embodiments, the texture formations can be formed over the full top surface of the top layer. In some embodiments, the texture formations may be formed over one or more regions of the top surface of the top layer, which may provide for the selection of one or more areas of foot sole to which stimulus is to be applied by the texture formations. The texture formations may provide a signal to a user of the orthosis, which may enhance proprioceptive awareness of terrain changes under the foot sole and increase motor response from muscles in the lower leg and foot. In some embodiments, the selection of one or more regions of the top surface of the top layer to include the texture formations may provide a means for targeting particular muscles, for example muscles of the lower leg or foot of the user.

According to some embodiments, the texture formations can be positioned on the top surface of the top layer in order to be associated with the forefoot region. The texture formations may cover the entire forefoot region, the medial forefoot region or the lateral forefoot region.

According to some embodiments, the texture formations can be positioned on the top surface of the top layer in order to be associated with the midfoot region. The texture formations may cover the entire midfoot region, the medial midfoot region or the lateral midfoot region.

According to some embodiments, the texture formations can be positioned on the top surface of the top layer in order to be associated with the rearfoot or heel region. The texture formations may cover the entire rearfoot or heel region, the medial rearfoot or heel region or the lateral rearfoot or heel region.

According to some embodiments, the texture formations are formed on the top surface of the top layer within one or more regions of the plantar foot sole and the remaining area of the top surface of the top layer can be formed as a smooth surface. According to some embodiments, the smooth surface can be formed on the same material from which the textured formations are formed. In some embodiments, the smooth surface can be formed from a material different from the material on which the textured formations are formed, however in these embodiments, the durometer of the different material can be substantially similar to that of the material used for the one or more regions having texture formations thereon. According to embodiments, the height of the smooth surface is substantially matched with the height with the texture formations.

According to embodiments, the texture formations can be formed in one or more configurations. For example, the texture formations can be one or more of dimples, ridges or other configurations as would be readily understood. In some embodiments, the ridges can be configured in one or more of a variety of different patterns. For example, the patterns can be one or more of a zigzag pattern, star shaped pattern, plus sign shaped pattern, sinusoidal shaped pattern, wave shaped pattern, chevon shaped pattern, herringbone shaped pattern or other ridge configuration or pattern. In some embodiments, the texture formations have a configuration that is symmetrical and in other configurations the texture formations have a non-symmetrical configuration.

According to some embodiments, the texture formations are configured with raised portions including abrupt changes in elevation thereof in one or more directions of the texture formations.

According to some embodiments, the texture formations are formed in a non-symmetrical configuration, wherein this configuration has an orientation. According to some embodiments, the orientation of the texture formations is dependent on an intended movement of a user during use of the orthosis. For example, the texture formations can be orientated perpendicular to the intended movement of the user of the orthosis, non-perpendicular to the intended movement of the user of the orthosis or at an orientation between parallel and perpendicular to the intended movement of the user of the orthosis. In some embodiments, the texture formations can be orientated at substantially a 45 degree angle with respect to the intended movement of the user of the orthosis.

According to some embodiments, the one or more regions of the top surface of the top layer are determined based on the movement of a user of orthosis or a direction of movement in which the user of the orthosis desired an increase in sensory feedback. For example, a user of the orthosis desiring an increase in sensory feedback during cutting movement or actions, for example changes in lateral movement of the user of the orthosis, may have texture formations formed in the medial forefoot region of the top surface of the top layer, wherein the texture formations have an orientation perpendicular to cutting movements. As another example, a user of the orthosis desiring an increase in sensory feedback during running, may have texture formations formed within the whole forefoot region of the top surface of the top layer, wherein the texture formations have an orientation perpendicular to the direction of running.

According to some embodiments, texture formations can be placed in a specific direction under the wearer's foot sole. Attention to the direction of movement of the user of the orthosis is desired. In some embodiments, the texture formations are configured in a zigzag pattern such that the orientation of the zigzag pattern is substantially perpendicular to the direction of locomotion or activity of the user of the orthosis. For example, if the wearer is running in a forward direction, the orientation of texture formations can be aligned in the mediolateral direction of this forward movement. If the wearer of the orthosis is participating in cutting activities, for example during the play of basketball, the orientation of the texture formations material may be aligned along an oblique axis to the cutting direction. As another example, the wearer of the orthosis can be a weight lifter, wherein the orientation of the texture formations can be along the length of the orthosis and may be located in a one or more regions of the rearfoot or heel region. This orientation and positioned of the texture formations may provide enhanced sensory input for that region in order to sense potential rotation of an ankle. As yet a further example, texture formations can be provided along the entire length of the insole for users of the orthosis that desire overall or global increases to sensory information from the plantar side of their feet. As a further example, having texture formations within the forefoot region and more particularly within the anterior end of the orthosis under the toe regions, may provide sensory input for a user with Parkinson's disease and this enhanced sensory input can provide a cue for initiating a subsequent step. In addition, a further example can be related to injury recovery, wherein the texture formations are located in a region within the medial midfoot region, wherein these texture formations may provide sensory input to aid with the stimulation of activity in the tibialis posterior muscle during walking, which may aid with the reduction of muscle fatigue or to trigger the activation of dormant musculature.

According to embodiments, due to the nature of mechanoreceptor activation, for example receptor response to skin deformation, stretch, pressure, the texture formations are configured to create an influx of stimuli as the user of the orthosis changes their center of pressure location as applied to the plantar side of their feet.

According to some embodiments, the texture formations have a height of between 1 mm and 5mm between the peak of the texture formation and the trough of the texture formations. According to some embodiments, the texture formations have a height of between 2 mm and 4 mm between the peak of the texture formation and the trough of the texture formations.

According to some embodiments, the height of the texture formations can at least in part be dependent on the hardness of the material forming the top layer. For example, a harder material may require a smaller height of texture formations. In some embodiments, the hardness of material of the top layer may be determined based on a desired level of sensory stimulation provided by the orthosis.

According to embodiments, the hardness or durometer of the material forming the top layer can vary between 20 dur and 60 dur, between 30 dur and 50 dur or other durometer which can be determined based on one or more of the intended movement of a user of the orthosis, the level of sensory input or one or more other parameters.

According to some embodiments, the top layer can be formed from a material having a thickness that is dependent on texture formations. For example, the top layer can have a thickness such that the top layer as measured from the bottom surface of the top layer to a peak of the texture formation can be between 2 and 4 times the distance from the bottom surface of the top layer to a trough of the texture formation. In some embodiments, the top layer has a thickness such that the top layer as measured from the bottom surface of the top layer to a peak of the texture formation can be between twice the distance from the bottom surface of the top layer to a trough of the texture formation.

According to some embodiments, the texture formations are configured in a zigzag pattern, wherein the raised indents have elevations of abrupt deflections in each direction. These changes in elevation of the texture formations can be configured to be symmetrical between deviations. For example, the distance between deviations can be selected between 3 mm and 10 mm or between 4 mm and 8 mm or at 7 mm. In addition, the distance between each horizontal zigzag pattern, namely the distance between each zigzag pattern, can be between 3 mm and 10 mm or between 4 mm and 8 mm or at 5 mm apart from each elevation.

According to some embodiments, the texture formations are formed in a zigzag pattern and the height between the peak and trough of the zigzag pattern can vary between 2 mm to 4 mm. The peak point of the zigzag pattern can be configured to be twice the height of the trough or non-raised portion from the bottom surface of the top layer. For example, if the highest point of the zigzag pattern, as measured from the bottom surface of the top layer, is 3 mm, the non-raised or trough would be 1.5 mm in height from the bottom surface of the top layer.

In some embodiments, the insole or the top layer or both can be formed from the same or different materials. The material can include one or more of ethyl vinyl acetate (EVA), other open cell foam or closed cell foam. The materials may be considered to include plastizotes, polyethylenes, or neoprenes or other suitable material as would be readily understood by a worker skilled in the art. According to some embodiments, the top layer can be formed from a material that is malleable, heat mouldable properties that is capable of providing the desired level of hardness or durometer and rigidity of the texture formations.

According to some embodiments, any orthosis according to embodiments of the present invention may be integrated into the footwear by manufactures or may be integrated into the design of sock liners for footwear.

Reference is now made to the figures, wherein in the medial portion is defined by reference numeral 30, the lateral portion is defined by reference numeral 40, the posterior portion is defined by numeral 10 and the anterior portion is defined by reference numeral 20. In addition, the contoured arch support is defined by reference numeral 110.

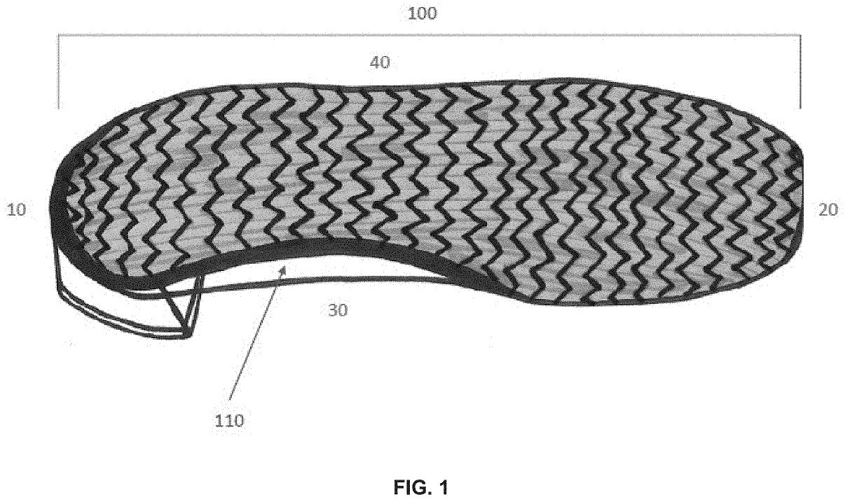

FIG. 1 illustrates a perspective view of a textured orthosis for a left foot according to embodiments. In this embodiment, the texture formations are formed over the full length 100 of the top layer of the orthosis.

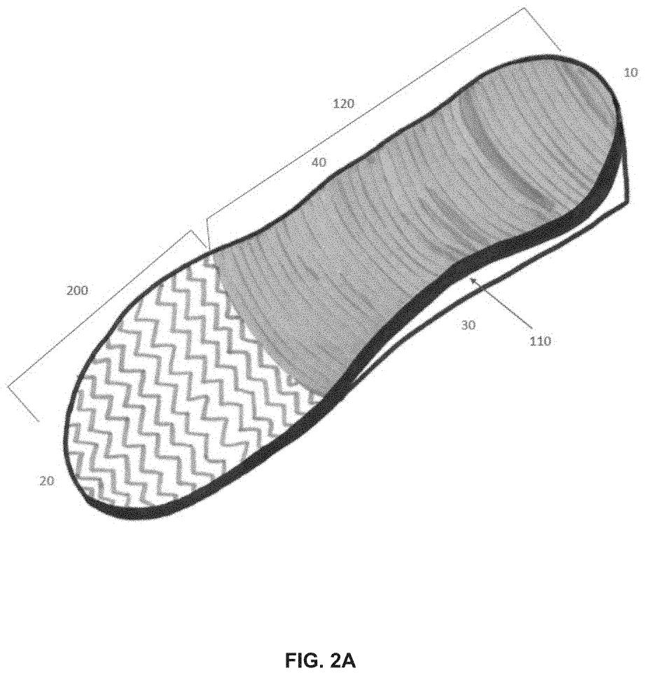

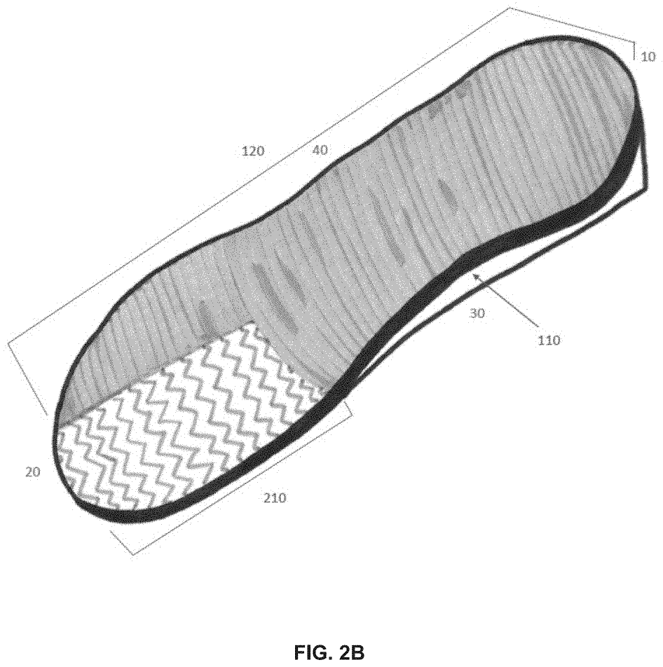

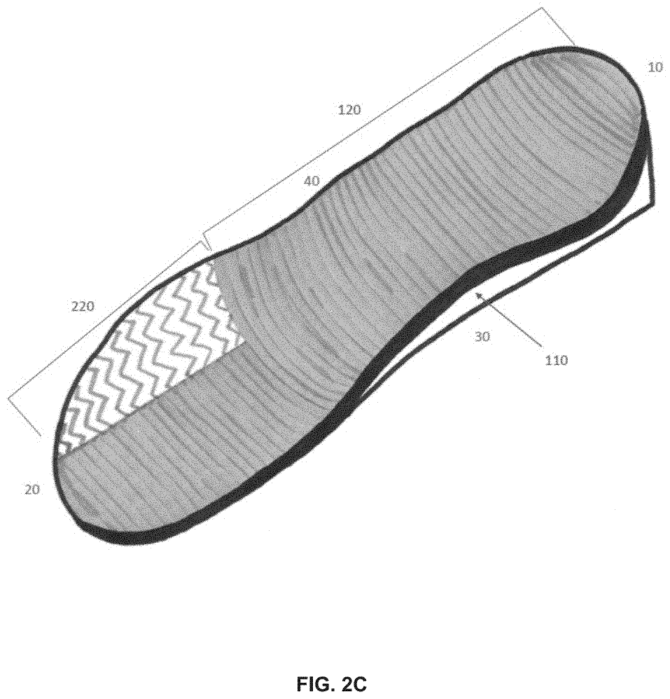

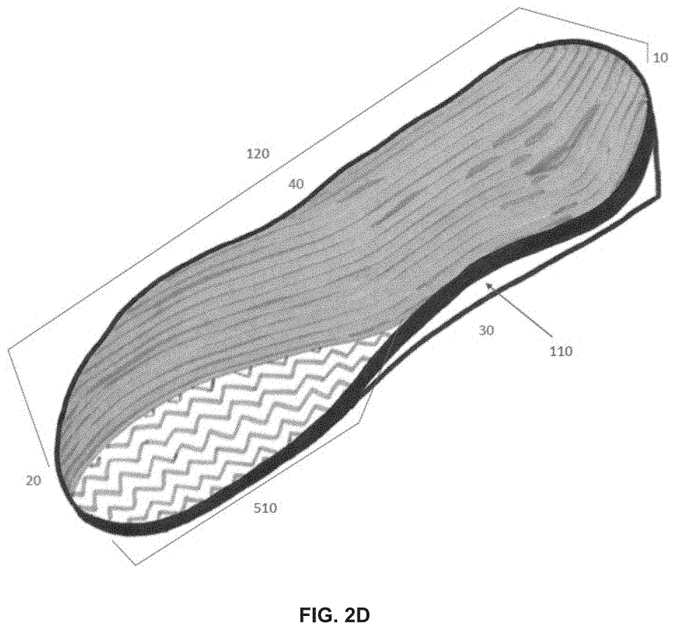

FIG. 2A illustrates a perspective view of a forefoot regionally textured orthosis for a right foot according to embodiments. In this embodiment, the texture formations are formed over the full forefoot region 200 of the top layer of the orthosis. The remaining portion 120 of the top layer is configured as a smooth surface.

FIG. 2B illustrates a perspective view of a forefoot regionally textured orthosis for a right foot according to embodiments. In this embodiment, the texture formations are formed over a medial forefoot region 210 of the top layer of the orthosis. The remaining portion 120 of the top layer is configured as a smooth surface.

FIG. 2C illustrates a perspective view of a forefoot regionally textured orthosis for a right foot according to embodiments. In this embodiment, the texture formations are formed over the lateral forefoot region 220 of the top layer of the orthosis. The remaining portion 120 of the top layer is configured as a smooth surface.

FIG. 2D illustrates a perspective view of a forefoot regionally textured orthosis for a right foot according to embodiments. In this embodiment, the texture formations are formed over a portion of the forefoot region 510 of the top layer of the orthosis. The remaining portion 120 of the top layer is configured as a smooth surface.

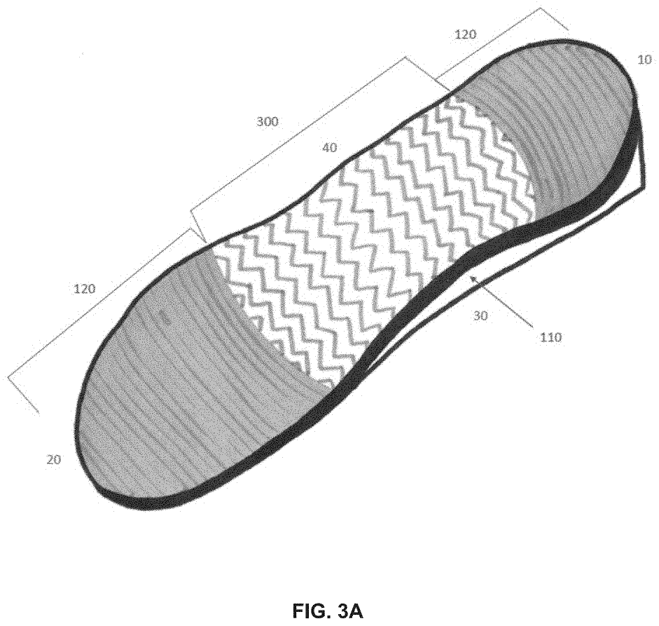

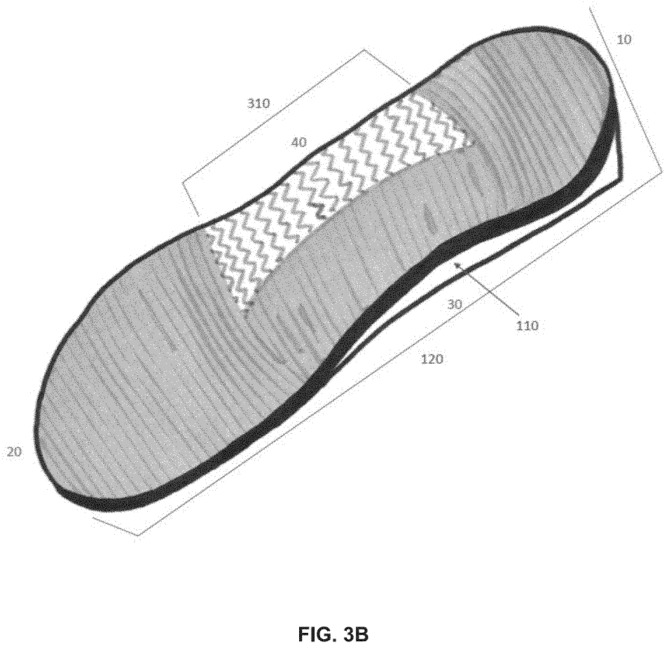

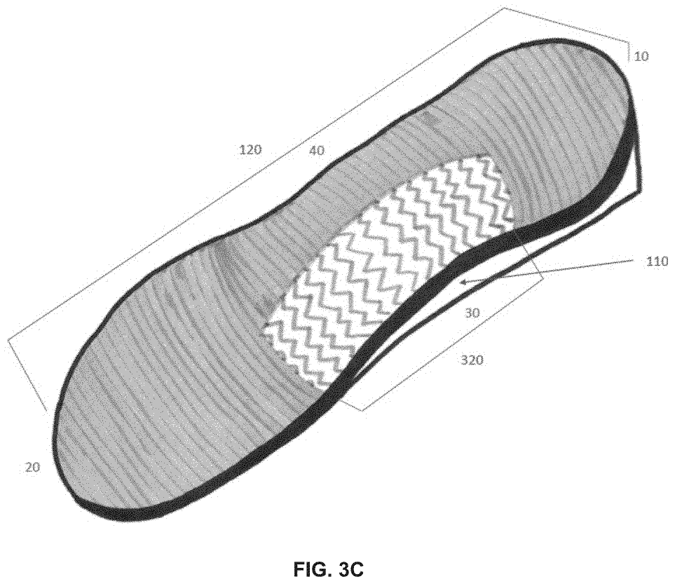

FIG. 3A illustrates a perspective view of a midfoot regionally textured orthosis for a right foot according to embodiments. In this embodiment, the texture formations are formed over the full midfoot region 300 of the top layer of the orthosis. The remaining portion 120 of the top layer is configured as a smooth surface.

FIG. 3B illustrates a perspective view of a midfoot regionally textured orthosis for a right foot according to embodiments. In this embodiment, the texture formations are formed over the lateral midfoot region 310 of the top layer of the orthosis. The remaining portions 120 of the top layer is configured as a smooth surface.

FIG. 3C illustrates a perspective view of a midfoot regionally textured orthosis for a right foot according to embodiments. In this embodiment, the texture formations are formed over the medial midfoot region 320 of the top layer of the orthosis. The remaining portions 120 of the top layer is configured as a smooth surface.

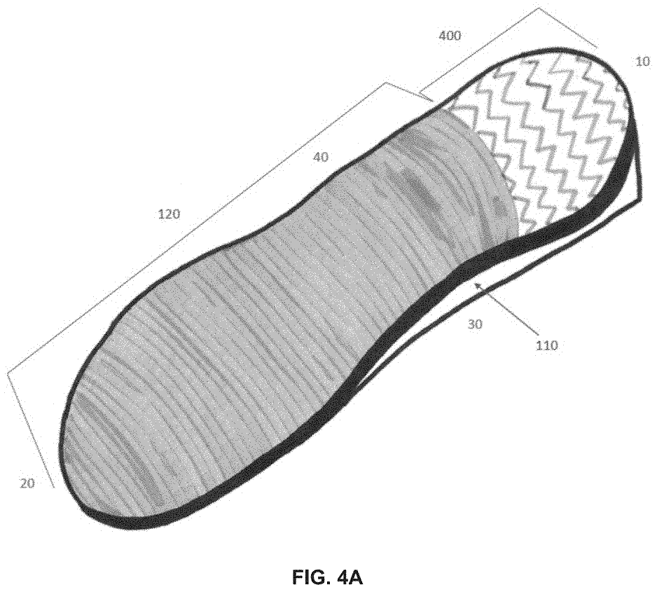

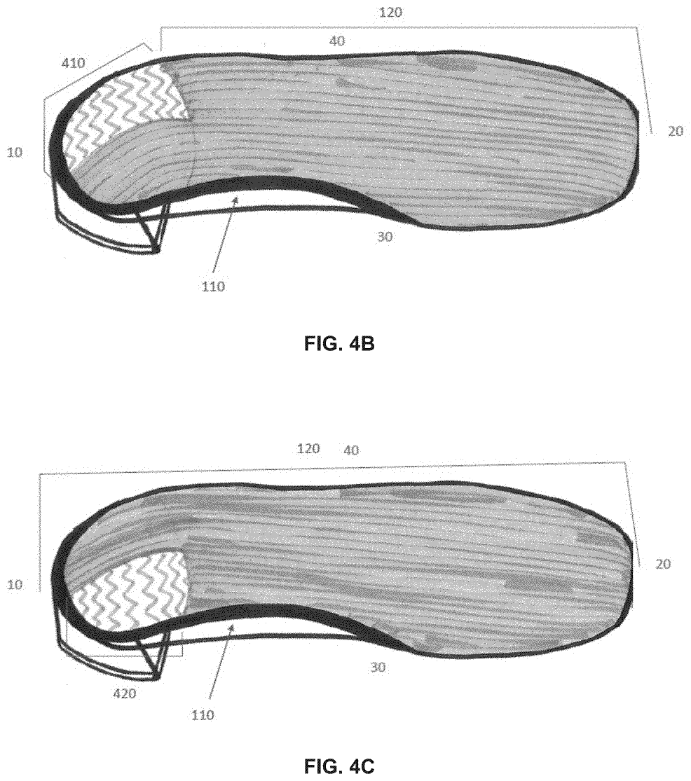

FIG. 4A illustrates a perspective view of a rearfoot regionally textured orthosis for a right foot according to embodiments. In this embodiment, the texture formations are formed over the full heel region 400 of the top layer of the orthosis. The remaining portions 120 of the top layer is configured as a smooth surface.

FIG. 4B illustrates a perspective view of a rearfoot regionally textured orthosis for a left foot according to embodiments. In this embodiment, the texture formations are formed over the lateral heel region 410 of the top layer of the orthosis. The remaining portions 120 of the top layer is configured as a smooth surface.

FIG. 4C illustrates a perspective view of a rearfoot regionally textured orthosis for a left foot according to embodiments. In this embodiment, the texture formations are formed over the medial heel region 420 of the top layer of the orthosis. The remaining portions 120 of the top layer is configured as a smooth surface.

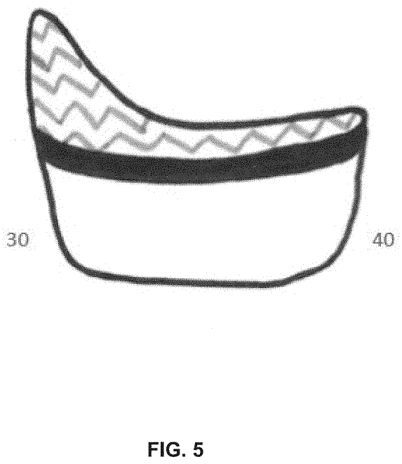

FIG. 5 illustrates a rear view of a rearfoot regionally textured orthosis for a right foot according to embodiments. In this embodiment, the heel region includes texture formations thereon, wherein the heel region is further contoured on the medial side of the orthosis.

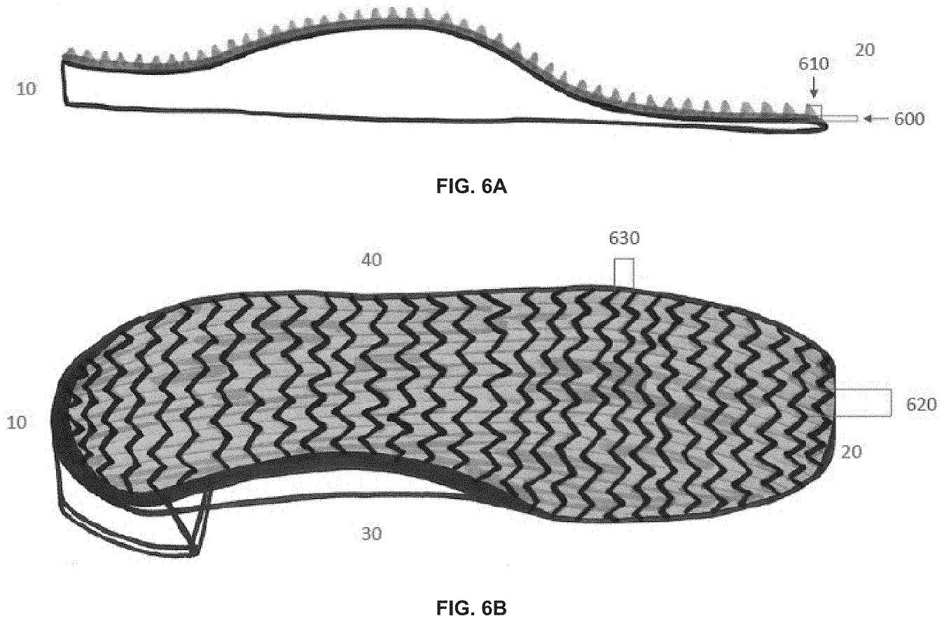

FIG. 6A illustrates a side view of a textured orthosis for a left foot according to embodiments. The thickness and height of the texture formations are clearly illustrated. The trough thickness 600 of the top layer and the peak thickness 610 are identified. In some embodiments, the peak thickness can be between 2 mm and 4 mm and the trough thickness is approximately half of the peak thickness. In some embodiments the trough thickness is 3 mm and the trough thickness is 1.5 mm.

FIG. 6B illustrates a perspective view of a textured orthosis for a left foot according to embodiments. As illustrated in FIG. 6B, in some embodiments, the texture formations are configured in a zigzag pattern, wherein the separation between the texture formations is defined by 630 and the peak to peak distance is defined by 620. In some embodiments, the separation 630 can be 5 mm and the peak to peak distance 620 can be 7 mm.

Based on the above discussion of an orthosis, in some instances the discussion relates to the right foot while in other instances the discussion relates to the left foot in accordance with embodiments of the present invention, it would be readily understood by a worker skilled in the art how to configure an orthosis for the other foot in accordance with embodiments of the present invention.

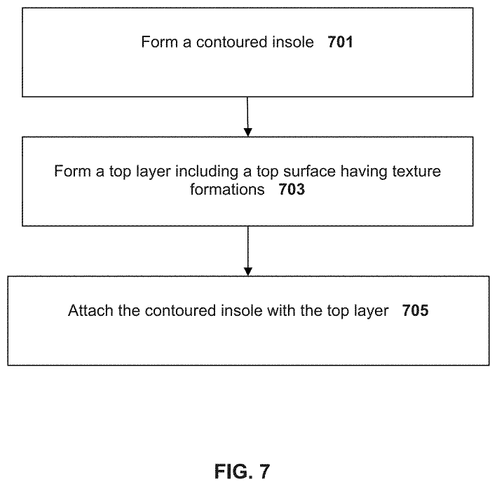

FIG. 7 illustrates a method an orthosis for improving plantar sensory input according to embodiments of the present invention. The method includes step 701 forming a contoured insole having a medial side portion, the medial side portion having a contoured arch support portion. The method further includes step 703 forming a top layer, the top layer including a top surface having texture formations formed within one or more regions associated with the top surface, the texture formations having an orientation, wherein the orientation is dependent on an intended movement of a user during use of the orthosis. In addition, the method includes step 705 attaching a bottom surface of the top layer to a top surface of the contoured insole.

In some embodiments, forming a top layer includes heat moulding the texture formations within the top layer. In some embodiments, one or more of the contoured insole and the top layer are formed using injection moulding, heat moulding, stamping, or other process. In some embodiments, forming one or more of the contoured insole and the top layer is performed using three dimensional printing. In some embodiments, one or more of the top layer and the contoured insole can be fabricated using computer aided design/computer aid manufacturing (CAD/CAM). In some embodiments, one or more of the top layer and the contoured insole can be fabricated via milling for example using a computer numerical control (CNC) machine.

According to some embodiments, wherein the orthosis is fabricated via direct milling, for example a CNC machine, the full orthosis including the top layer and the contoured insole can be fabricated from a single component, which is either a single material or a preformed block of material. In some embodiments, the preformed block of material can be formed from two or more materials, wherein each material is selected, e.g. to have a particular durometer, to function as the contoured insole or into which the texture formations are to be milled.

It will be appreciated that, although specific embodiments of the technology have been described herein for purposes of illustration, various modifications may be made without departing from the scope of the technology. The specification and drawings are, accordingly, to be regarded simply as an illustration of the invention as defined by the appended claims, and are contemplated to cover any and all modifications, variations, combinations or equivalents that fall within the scope of the present invention.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

We claim:

1. An orthosis comprising:
a contoured insole having a medial side portion, the medial side portion having a contoured arch support portion; and
a top layer having a bottom surface coupled to a top surface of the contoured insole, the top layer including a top surface having texture formations formed within one or more regions associated with the top surface, the texture formations having an orientation, wherein the orientation is dependent on an intended movement of a user during use of the orthosis.

2. The orthosis according to claim 1, wherein the texture formations are configured as one or more of dimples or ridges.

3. The orthosis according to claim 1, wherein the texture formations are configured as ridges defining one or more patterns on the top surface of the top layer.

4. The orthosis according to claim 3, wherein the one or more patterns include a zigzag pattern, star shaped pattern, plus sign pattern, sinusoidal shaped pattern, a wave shaped pattern, chevron shaped pattern and a herringbone shaped pattern.

5. The orthosis according to claim 1, wherein a thickness of the top layer at a peak of a texture formation is twice a thickness of the top layer at a trough of the texture formation.

6. The orthosis according to claim 1, wherein the one or more regions include one or more of a forefoot region, a midfoot region and a heel region.

7. The orthosis according to claim 1, wherein each of the forefoot region, the midfoot region and the heel region are defined by two regions including a medial regarding and a lateral region.

8. The orthosis according to claim 1, wherein the orientation of the texture formations is perpendicular to the intended movement of the user during use of the orthosis.

9. The orthosis according to claim 1, wherein the orientation of the texture formations is parallel to the intended movement of the user during use of the orthosis.

10. The orthosis according to claim 1, wherein the orientation of the texture formations is at an angle of 45 degrees with respect to the intended movement of the user during use of the orthosis.

11. The orthosis according to claim 1, wherein the intended movement of the user during use of the orthosis is a cutting movement and the one or more regions include a lateral forefoot region.

12. The orthosis according to claim 1, wherein the intended movement of the user during use of the orthosis is a running movement and the one or more regions include a medial forefoot region and a lateral forefoot region.

13. The orthosis according to claim 1, wherein the orthosis is a custom-made orthosis or integrated into a sock liner.

14. The orthosis according to claim 1, wherein the orthosis is a prefabricated orthosis having a standard design for one or more selected foot configurations.

15. The orthosis according to claim 14, wherein the selected foot configurations are dependent on a foot size and a level of arch support.

16. A method for making an orthosis, the method comprising:
forming a contoured insole having a medial side portion, the medial side portion having a contoured arch support portion;
forming a top layer, the top layer including a top surface having texture formations formed within one or more regions associated with the top surface, the texture formations having an orientation, wherein the orientation is dependent on an intended movement of a user during use of the orthosis; and
attaching a bottom surface of the top layer to a top surface of the contoured insole.

17. The method according to claim 16, wherein forming one or more of the contoured insole and the top layer is performed using three dimensional printing.

18. The method according to claim 16, wherein forming the top layer includes forming the texture formations using heat moulding.

19. The method according to claim 16, wherein the texture formations are formed using direct milling.
